(19) United States
(12) Patent Application Publication
(10) Pub. No.: US 2019/0099216 A1
(43) Pub. Date: Apr. 4, 2019

(54) ELECTROSURGICAL INSTRUMENT

(71) Applicant: Spiration, Inc., d.b.a. Olympus Respiratory America, Redmond, WA (US)

(72) Inventors: Brandon J. Shuman, Kirkland, WA (US); Peter Lambe, Renton, WA (US); Chenhao Fu, Renton, WA (US); Zoie R. Engman, Redmond, WA (US); Cassandra A. Saleira, Redmond, WA (US)

(21) Appl. No.: 16/087,314

(22) PCT Filed: Feb. 22, 2017
(86) PCT No.: PCT/US2017/018826

§ 371 Date: Sep. 21, 2018

Related U.S. Application Data
(60) Provisional application No. 62/307,291, filed on Mar. 11, 2016.
(60) Provisional application No. 62/311,048, filed on Mar. 21, 2016.

(51) Int. Cl. A61B 18/14; A61B 18/12

(52) U.S. Cl. CPC A61B 18/1477; A61B 2017/00867; A61B 18/1206

(57) ABSTRACT

An electrosurgical instrument includes a needle configured as a first electrode and a coil extending through the needle and configured as a second electrode. The coil movable relative to the needle, and as the needle and the coil are inserted into tissue and energized with an electrical energy source, the needle and the coil apply current to the tissue to coagulate the tissue.

### RELATED APPLICATION

This application claims the benefit of U.S. Provisional Patent Application No. 62/307,291, filed on Mar. 11, 2016 and U.S. Provisional Patent Application No. 62/311,048, filed on Mar. 21, 2016.

The contents of above applications are incorporated herein by reference in their entirety.

### FIELD

The present relates to an electrosurgical instrument for treating tissue.

### BACKGROUND

The statements in this section merely provide background information related to the present disclosure and may not constitute prior art.

Certain electrosurgical instruments used for treating tissue generally include a guide catheter and an applicator inserted through the catheter. These electrosurgical instruments are usually inserted into a body lumen to place the distal end of the applicator at a desired location. The applicator generally includes one or more electrodes at the distal end. Such electrodes emit a radiofrequency (RF) electric current to surrounding tissue to coagulate or ablate the tissue. Monopolar electrosurgical instruments only require one electrode that interacts with a neutral electrode, which is likewise connected to the body of a patient. A bipolar electrosurgical instrument typically includes an applicator with two electrodes (a distal electrode and a proximal electrode). A RF voltage with different potentials is applied to such bipolar instruments so that a current passes from one electrode to the other electrode through the tissue, thereby heating the tissue to coagulate or ablate the tissue.

During the treatment of tissue with the above-described electrosurgical instruments, visualization of the electrodes would be beneficial. Further, during the treatment process, heated tissue may disengage from an electrode.

Accordingly, there is a need in the art for an electrosurgical instrument with electrodes that enhance treatment of tissue and enhance visualization of the electrodes during the treatment process.

### SUMMARY

The present invention provides an electrosurgical instrument for treating tissue, for example, ablating or coagulating tissue.

Accordingly, pursuant to one aspect of the present invention, an electrosurgical instrument includes a needle configured as a first electrode and a coil extending through the needle and configured as a second electrode. The coil movable relative to the needle, and as the needle and the coil are inserted into tissue and energized with an electrical energy source, the needle and the coil apply current to the tissue to coagulate the tissue.

This aspect may be further characterized by one or any combination of the features described herein, such as: the coil is electrically insulated from the needle; the needle and/or the coil includes one or more echogenic features on the exterior of the needle to aid visualizing the needle when inserted into tissue; the needle is visualized when the needle is energized in a monopolar mode; the one or more echogenic features is one or more etched bands; the one or more etched bands is a plurality of spaced apart etched bands; the coil is made of a shape memory alloy; a distal portion of the coil returns to a first coiled state when the coil is heated; as the distal portion of the coil returns to the first coiled state, movement of the coil enables visualizing the coil ultrasonically; as the coil returns to the first coiled state, the coiled portion of the coil re-engages tissue that previously shrunk away from the coil when the tissue was heated; and the coil has a first coiled distal portion and a second non-coiled portion, the non-coiled portion contracting when the coil is heated to apply tension on the first coiled portion inserted into tissue.

Accordingly, pursuant to another aspect of the present invention, a method of treating tissue includes one or more of the following steps: positioning an applicator in a passageway, extending a needle through the applicator, the needle being a first electrode, piercing the needle into tissue, advancing a coil through the needle, a distal portion of the coil piercing into the tissue, the coil being a second electrode, and energizing the needle and the coil with an electrically energy source in a bipolar mode to coagulate the tissue.

The method may be further characterized by one or any combination of the following features: the needle includes one or more echogenic features on the exterior of the needle; energizing the needle with the electrical energy source in a monopolar mode to aid visualizing the needle; the coil is made of a shape memory alloy; heating the coil such that a distal portion of the coil returns to a first coiled state when the coil is heated; as the distal portion of the coil returns to the first coiled state, movement of the coil enables visualizing the coil ultrasonically; as the coil returns to the first coiled state, the coiled portion of the coil re-engages tissue that previously shrunk away from the coil when the tissue was heated; and the coil has a first coiled distal portion and a second non-coiled portion, the non-coiled portion contracting when the coil is heated to apply tension on the first coiled portion inserted into tissue.

Accordingly, pursuant to yet another aspect of the present invention, a system for treating tissue includes an energy source, a needle connected to the energy source, the needle being a first electrode, and a coil extending through the needle and connected to the energy source. The coil is a second electrode and is movable relative to the needle. As the needle and the coil are inserted into tissue and energized with the electrical energy source, the needle and the coil apply current to the tissue to coagulate the tissue.

This aspect of the invention may be further characterized by one or any combination of the following features, such as: the coil is electrically insulated from the needle; the needle includes one or more echogenic features on the exterior of the needle to aid visualizing the needle when inserted into tissue; the needle is visualized when the needle is energized in a monopolar mode; the one or more echogenic features is one or more etched bands; the coil is made of a shape memory alloy; a distal portion of the coil returns to a first coiled state when the coil is heated; as the distal portion of the coil returns to the first coiled state, movement of the coil enables visualizing the coil ultrasonically; as the coil returns to the first coiled state, the coiled portion of the coil re-engages tissue that previously shrunk away from the coil when the tissue was heated; and the coil has a first coiled distal portion and a second non-coiled portion, the non-coiled portion contracting when the coil is heated to apply tension on the first coiled portion inserted into tissue.

Further features, advantages, and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, emphasis instead being placed upon illustrating the principles of the invention. In the drawings:

FIG. 1 illustrates an overview of a system for treating tissue in accordance with the principles of the present invention;

FIG. 2 illustrates a handle for the system shown in FIG. 1;

FIG. 3 illustrates a close-up view of a sheath, a needle, and a coil for the system shown in FIG. 1;

FIG. 4 illustrates the needle penetrating tissue;

FIG. 5A illustrates advancement of the coil into the tissue;

FIG. 5B illustrates energizing the coil when inserted into the tissue;

FIG. 6 illustrates an alternative coil in accordance with the principles of the present invention;

FIG. 7 illustrates alternative use of the coil in accordance with the principles of the present invention;

FIG. 8 illustrates yet another coil in accordance with the principles of the present invention; and

FIG. 9 is a side view showing a process for using the electrosurgical device shown in FIG. 3 in accordance with the principles of the present invention.

### DETAILED DESCRIPTION

The following description is merely exemplary in nature and is not intended to limit the present disclosure, application, or uses.

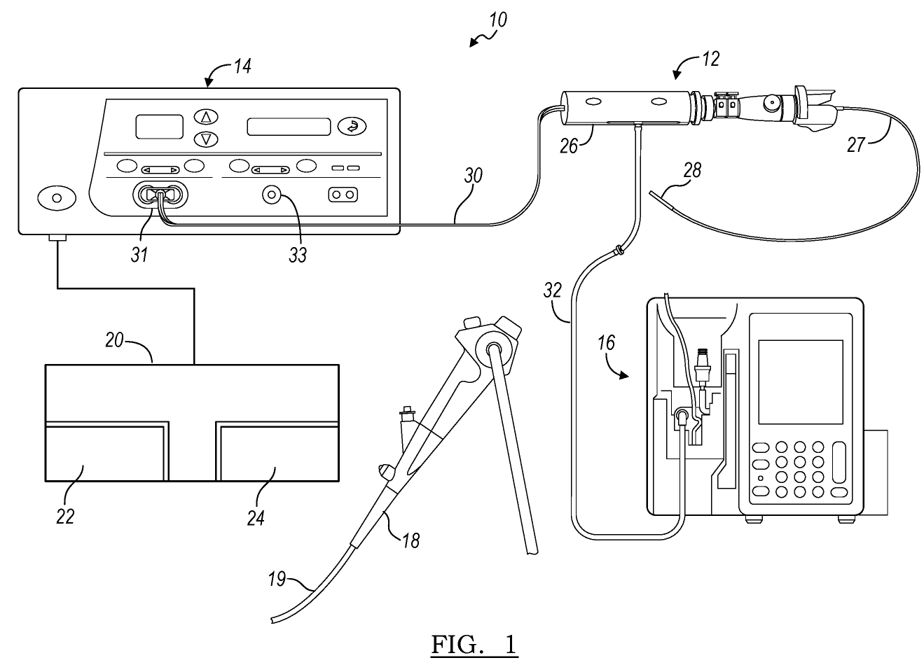

Referring now to FIG. 1, a system for treating tissue in an anatomical region of a patient is illustrated therein and designated at 10. The system 10 is a bipolar radio frequency (RF) system for treating tissue in a patient. Specifically, the system 10 is employed for coagulation and ablation of soft tissue during percutaneous and endoscopic, including bronchoscopic, surgical procedures, such as, for example, partial or complete ablation of organ lesions.

In some arrangements, the system 10 includes an applicator 12, an electrosurgical RF generator 14, an infusion pump 16, and a bronchoscope 18. The applicator 12 electrically communicates with the generator 14 though a lead 30. The lead 30 is connected to a generator outlet 31 when the system is operated in a bipolar mode. Alternatively, the system 10 can be operated in a monopolar mode when the lead 30 is connected to an outlet 33 with an adapter as necessary. The applicator 12 is further connected to the infusion pump 16 with a tube 32 that facilitates the flow of liquid, for example saline solution, from the pump 16 to the applicator 12.

The generator 14 can be operated with the use of a foot operated unit 20 electrically connected to the generator 14. The foot operated unit 20 includes a pedal 22 that instructs the generator 14 to apply a RF potential to electrodes (described below) to cut or ablate tissue and a pedal 24 that instructs the generator 14 to apply a lower RF potential to the electrodes to coagulate tissue.

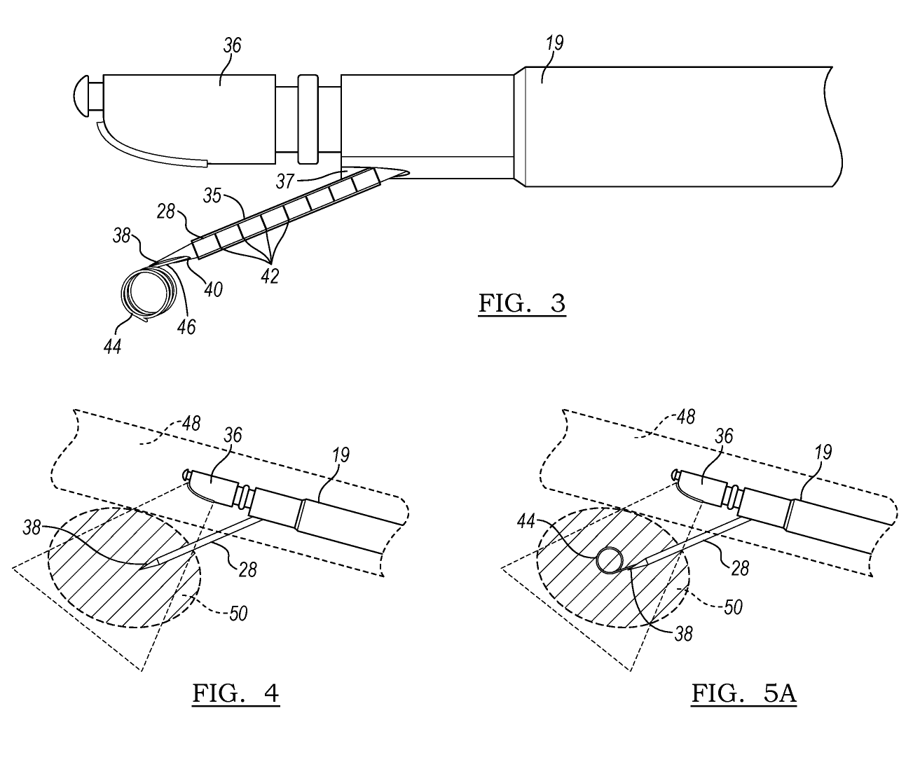

The bronchoscope 18 includes a sheath 19. At a distal end 36 (FIG. 3) of the sheath 19 is an opening 37. The applicator 12 includes a handle 26 and a needle 28 attached to a distal end of an extension or shaft 27. As such, in certain procedures, the needle 28 and the extension 27 are inserted into the bronchoscope 18 so that the needle 28 exits the distal end 36 through the opening 37.

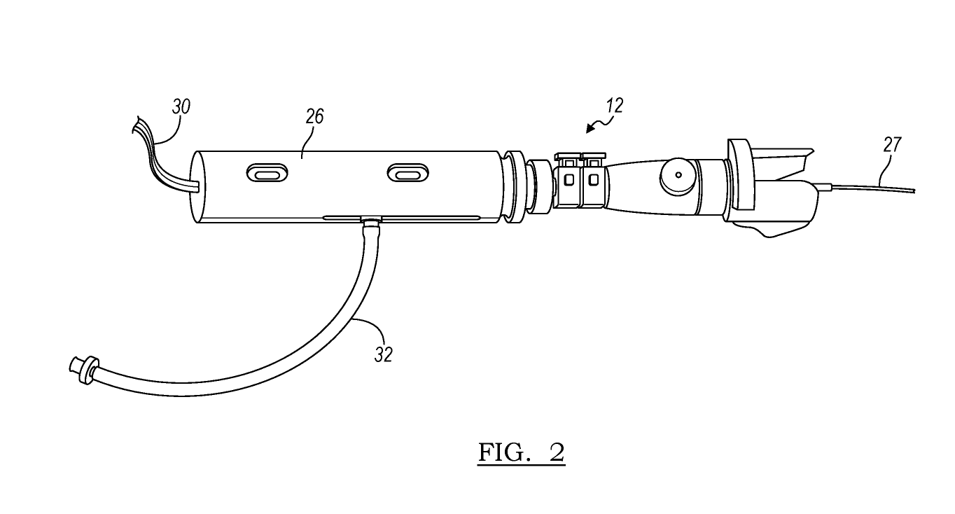

Referring also to FIGS. 2 and 3, the applicator 12 further includes a coil 44 that extends through the extension 27 and the needle 28 and exits an opening 40 of the needle 28. The needle 28 includes a layer of insulation 35, and the coil 44 includes a layer of insulation 46 up to the coiled end of the coil 44 to electrically isolate the needle 28 and the coil 44. Accordingly, in this arrangement, the needle 28 operates as a proximal electrode and the coil 44 operates as a distal electrode when the system 10 is operated in a bipolar mode.

The needle 28 also includes a tip 38 for piercing tissue and a set of echogenic features 42. In the arrangement shown in FIG. 3, the echogenic features are spaced apart bands that have been etched or scribed, for example, by a laser, on the outer surface of the needle 28. These bands 42 enhance ultrasonic visualization of the needle 28 during a procedure. Specifically, during the penetration of the needle 28 into tissue, only the needle 28 (that is, not the coil 44) is energized in a monopolar mode (for example, with the patient grounded to a patient pad to complete the circuit) with the generator 14 at a low power level. This causes the tissue to vibrate so that it can be visualized ultrasonically. The echogenic features 42 further enhance the ultrasonic visualization of the needle 28.

Referring further to FIGS. 4, 5A and 5B, there is illustrated a procedure for using the system 10 during, for example, bronchoscopy. Initially, a physician advances the sheath 19 of the bronchoscope 18 through a passageway, for example, an airway 48, until the distal end 36 is positioned near the tissue 50 to be treated. The physician then inserts the needle 28 into the sheath 19 and advances the needle 28 until the needle 28 exits the opening 37 and penetrates into the tissue 50 with the tip 38. While being visualized ultrasonically as described above, the needle 28 is positioned at a desired location in the tissue 50 (FIG. 4). Next the physician advances the coil 44 through the needle 28 until it exits the opening 40. The physician continues to advance the coil 44 to the desired location (FIG. 5A). Placement of the coil 44 can be visualized ultrasonically as well.

As shown in FIG. 5B, the coil 44 corkscrews into the tissue 50 with a diameter d2. The extent of penetration of the needle 28 (that is, the distance from the distal end 36 of the sheath 19 to the tip 38 of the needle 28) is d1, and the distance from the coiled portion of the coil 44 to the tip 38 of the needle 28 is d3. After the coil 44 has been deployed and prior to activating the electrodes (that is, the needle 28 and the coil 44), the needle 28 is retracted proximally while the coil 44 and the sheath 19 (and hence the distal end 36) are held in place so that dl decreases and d3 increases.

To energize the electrodes (the needle 28, the coil 44) for coagulating the tissue 50, the physician sets the generator 14 to a desired power level and pushes the pedal 24 of the foot unit 20 to apply a RF potential to the electrodes. As such, RF electrical current passes between the needle 28 and the coil 44 through the tissue 50 as indicated by the arrows 52. The level of RF electrical current is set by the physician to control the desired extent of the coagulation region 54 in the tissue 50. To ablate or cut tissue, the physician pushes the pedal 22 of the foot unit 20 to apply a higher RF potential to the electrodes. Note that anytime during the procedure, the physician can activate the infusion pump 16 to supply saline solution to the applicator 12 so that the saline solution flows through the needle 27 and the extension 28 to the location of interest in the tissue 50. The saline solution is employed to cool the electrodes (the needle 28 and/or the coil 44) and to prevent dehydration of the tissue 50.

After treatment of the tissue 50 is completed, the physician turns off the generator 14, moves the needle 28 forward to the position relative to the coil prior to coil deployment. Then, the coil 44 is retracted into the needle 28. Then, the needle 28 and the coil 44 are retracted into the sheath 19 within the bronchoscope 18, and withdraws the bronchoscope 18 from the patient.

The needle 28 is made from any suitable material, such as, for example, stainless steel. The coil 44 is also made from any suitable material that enables it to be corkscrewed into tissue. In various arrangements, all or a portion of the coil 44 is made from a shape memory alloy, such as NiTi for either its super-elastic properties or its shape memory features. When the coil 44 is made of shape memory alloy and is implemented for its shape memory properties, the portion of the coil made of shape memory alloy has a first configuration or state and a second configuration or state. Accordingly, when the coil 44 is in one of the states and then heated, the coil returns to the other pre-defined configuration. Subsequently, if the coil 44 is cooled, it returns to the configuration it had when unheated.

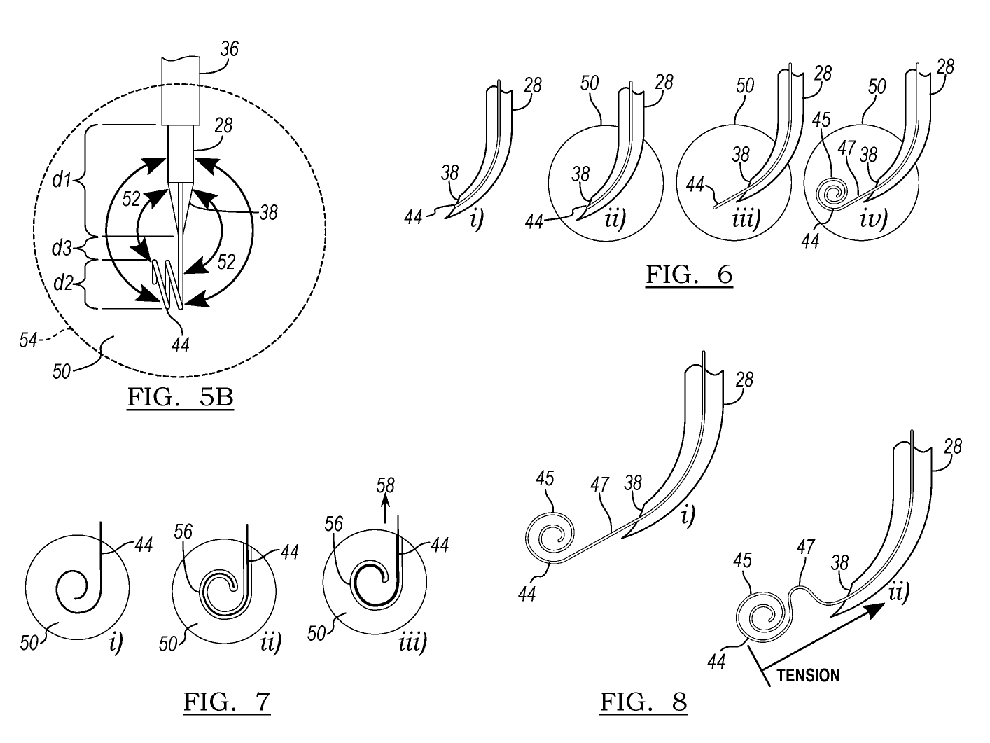

An example of the use of the coil 44 made of shape memory alloy is shown in FIG. 6. In step i), the coil 44 resides in the needle in an uncoiled state. In step ii), the needle 28 as well as the uncoiled coil 44 is inserted into tissue 50. In step iii), the uncoiled coil 44 is advanced into the tissue 50 and the needle 28 is retracted. And in step iv), the coil 44 is energized by, for example, the generator 14 to heat the coil 44 so that a portion 45 coils into the tissue 50 while a portion 47 remains uncoiled. That is, when heated the portion 45 returns to its previously defined coiled shape. As this occurs, the portion 45 causes significant movement in the tissue 50, which aids observation of the tissue by ultrasound.

In a particular configuration, the coil 44 shown in FIG. 6 is made of NiTi with a transition temperature of about 37° C. Hence, when heated about this temperature, the portion 45 returns to its austenitic state, which in this case is a pre-defined coiled state. After treatment of the tissue 50, the coil 44 is cooled below the transition temperature so that the coil returns to its martensitic state. Since the coil portion 45 in its martensitic state is less stiff than when it is in its austenitic state, the coil 44 can be easily retracted from the tissue and through the needle 28 and the extension 27.

Turning now to FIG. 7, there is shown another alternative use of the coil 44. In step i), the coil 44 is advanced so that it corkscrews into the tissue 50. In step ii), the coil 44 along with the needle 28 is energized to coagulate or ablate the tissue 50. During treatment of the tissue 50, the tissue may shrink and disengage from the coil 44 as indicated by the outline 56. To re-engage the coil 44 with the tissue 50, tension is applied in the direction of arrow 58 to the coil 44 so that it re-engages with the tissue 50 as indicated in step iii).

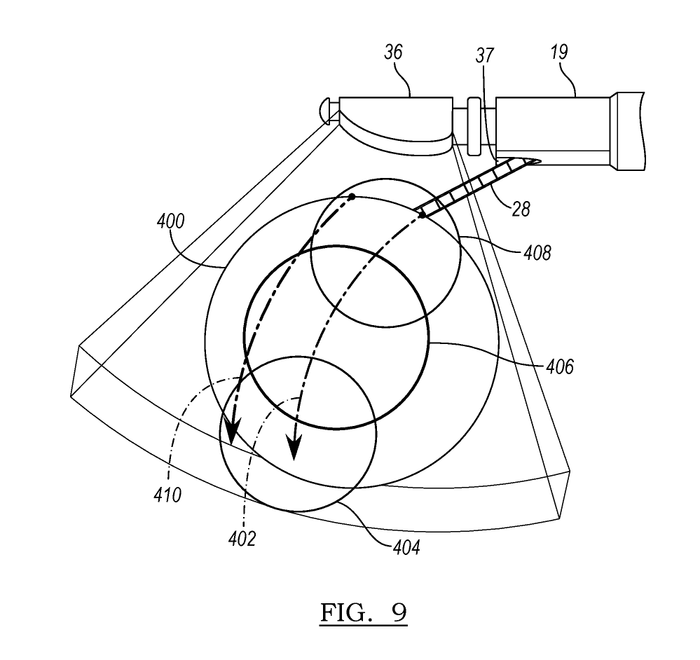

Tension can be applied to the coil 44 by any suitable mechanism. For example, the physician can simply pull on and/or twist the coil 44. Or the coil can be pre-loaded with a tension through the use of a spring. Alternatively, as shown in FIG. 8, a portion 47 of the coil 44 can be made of shape memory alloy so that when the temperature of the coil 44 is below the transition temperature of the shape memory alloy, the portion 47 is straight while the portion 45 is coiled into tissue and the needle 28 is retracted as shown in step i). In step ii), the coil 44 is heated so that the temperature of the portion 47 rises above the transition temperature of the selected shape memory allow. As this occurs, the portion 47 returns to its pre-defined curved state which, in turn, which creates tension in the portion 45 as it is pulled against the tissue 50.

Turning now to FIG. 9, there is shown the needle 28 being employed in an ablation process. The needle 28 exits the opening 37 at the distal end 36 of the sheath 19. The needle 28 is inserted into a target region 400 in the interior region of a patient such that the operator is provided with a consistent method of using the needle 28 in combination the aforementioned coil 44 for coagulating tissue and creating a reproducible coagulation volume in the target region 400.

In addition, FIG. 9 shows an ablation process or pathline technique that takes into account issues associated with over-penetration and potential saline drainage. The pathline technique maximizes the efficiency of coagulation by ensuring that the operator moves the needle 28 and the coil 44 as tissue conductivity decreases, effectively creating a larger coagulation volume then attempting to coagulate tissue in a single location.

More specifically, the needle 28 utilizes the needle track created during penetration into the target 400 region to coagulate tissue along a needle path 402, starting at the deepest location 404 initially to seal the needle path. The operator retracts the needle 28 to, for example, a middle location 406 and coagulates tissue in this region if desired, and then retracts the needle 28 to the proximal edge 408 of the target region 400 and coagulates tissue in this region if desired. Hence, the operator is procedurally able to coagulate tissue at locations along the needle path 402 that are closer to the initial penetration location. The pathline technique creates a more consistent and reproducible coagulation volume, while reducing the limitations of high tissue impedance. Once a first portion of the target region has been coagulated, the operator may reinsert the needle 30 and the coil 44 along a second path 410 and coagulate in the same manner as the first path 402.

The description of the invention is merely exemplary in nature and variations that do not depart from the gist of the invention are intended to be within the scope of the invention. Such variations are not to be regarded as a departure from the spirit and scope of the invention.

What is claimed is:

1. An electrosurgical instrument comprising:
a needle configured as a first electrode; and
a coil extending through the needle and configured as a second electrode, the coil being movable relative to the needle,
wherein as the needle and the coil are inserted into tissue and energized with an electrical energy source, the needle and the coil apply current to the tissue to coagulate the tissue.

2. The electrosurgical device of claim 1 wherein the coil is electrically insulated from the needle.

3. The electrosurgical device of claim 1 wherein the needle includes one or more echogenic features on the exterior of the needle to aid visualizing the needle when inserted into tissue.

4. The electrosurgical device of claim 3 wherein the needle is visualized when the needle is energized in a monopolar mode.

5. The electrosurgical device of claim 3 wherein the one or more echogenic features is one or more etched bands.

6. The electrosurgical device of claim 5 wherein the one or more etched bands is a plurality of spaced apart etched bands.

7. The electrosurgical device of claim 1 wherein the coil is made of a shape memory alloy.

8. The electrosurgical device of claim 7 wherein a distal portion of the coil returns to a first coiled state when the coil is heated.

9. The electrosurgical device of claim 8 wherein as the distal portion of the coil returns to the first coiled state, movement of the coil enables visualizing the coil ultrasonically.

10. The electrosurgical device of claim 8 wherein as the coil returns to the first coiled state, the coiled portion of the coil re-engages tissue that previously shrunk away from the coil when the tissue was heated.

11. The electrosurgical device of claim 7 wherein the coil has a first coiled distal portion and a second non-coiled portion, the non-coiled portion contracting when the coil is heated to apply tension on the first coiled portion inserted into tissue.

12. A method of treating tissue, the method comprising:
positioning an applicator in a passageway;
extending a needle through the applicator, the needle being a first electrode;
piercing the needle into tissue;
advancing a coil through the needle, a distal portion of the coil piercing into the tissue, the coil being a second electrode; and
energizing the needle and the coil with an electrically energy source in a bipolar mode to coagulate the tissue.

13. The method of claim 12 wherein the needle includes one or more echogenic features on the exterior of the needle.

14. The method of claim 13 further comprising energizing the needle with the electrical energy source in a monopolar mode to aid visualizing the needle.

15. The method of claim 12 wherein the coil is made of a shape memory alloy.

16. The method of claim 15 further comprising heating the coil such that a distal portion of the coil returns to a first coiled state when the coil is heated.

17. The method of claim 15 wherein as the distal portion of the coil returns to the first coiled state, movement of the coil enables visualizing the coil ultrasonically.

18. The method of claim 15 wherein as the coil returns to the first coiled state, the coiled portion of the coil re-engages tissue that previously shrunk away from the coil when the tissue was heated.

19. The method of claim 15 wherein the coil has a first coiled distal portion and a second non-coiled portion, the non-coiled portion contracting when the coil is heated to apply tension on the first coiled portion inserted into tissue.

20. A system for treating tissue comprising:
an energy source; and
a needle connected to the energy source, the needle being a first electrode;
a coil extending through the needle and connected to the energy source, the coil being a second electrode and being movable relative to the needle,
wherein as the needle and the coil are inserted into tissue and energized with the electrical energy source, the needle and the coil apply current to the tissue to coagulate the tissue.

21. The system of claim 20 wherein the coil is electrically insulated from the needle.

22. The system of claim 20 wherein the needle includes one or more echogenic features on the exterior of the needle to aid visualizing the needle when inserted into tissue.

23. The system of claim 22 wherein the needle is visualized when the needle is energized in a monopolar mode.

24. The system of claim 22 wherein the one or more echogenic features is one or more etched bands.

25. The system of claim 20 wherein the coil is made of a shape memory alloy.

26. The system of claim 25 wherein a distal portion of the coil returns to a first coiled state when the coil is heated.

27. The system of claim 26 wherein as the distal portion of the coil returns to the first coiled state, movement of the coil enables visualizing the coil ultrasonically.

28. The system of claim 26 wherein as the coil returns to the first coiled state, the coiled portion of the coil re-engages tissue that previously shrunk away from the coil when the tissue was heated.

29. The system of claim 25 wherein the coil has a first coiled distal portion and a second non-coiled portion, the non-coiled portion contracting when the coil is heated to apply tension on the first coiled portion inserted into tissue.
